(19) United States
(12) Patent Application Publication
(10) Pub. No.: US 2009/0170772 A1
(43) Pub. Date: Jul. 2, 2009

(54) INTERLEUKIN PRODUCTION REGULATOR, PHARMACEUTICAL COMPOSITION OR FOOD COMPRISING THE INTERLEUKIN PRODUCTION REGULATOR, AND METHOD FOR PRODUCTION OF THE INTERLEUKIN PRODUCTION REGULATOR

(75) Inventors: Noriyuki Iwabuchi, Kanagawa (JP); Kanetada Shimizu, Kanagawa (JP); Noritoshi Takahashi, Kanagawa (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: MORINAGA MILK INDUSTRY CO., LTD., Tokyo (JP)

(21) Appl. No.: 12/295,091

(22) PCT Filed: Mar. 8, 2007
(86) PCT No.: PCT/JP2007/054567

§ 371 Date: Sep. 29, 2008

(30) Foreign Application Priority Data
Mar. 31, 2006 (JP) 2006-099905

(51) Int. Cl. A61K 38/16; C12P 21/00; C07K 14/00; A61P 3/10

(52) U.S. Cl. 514/12; 435/71.2; 530/350

(57) ABSTRACT

An object is to provide an interleukin production regulator which can be used for prevention, treatment or recurrence prevention of a gastrointestinal disease or an autoimmune disease accompanied by an inflammation as the main symptom, which is highly safe, and which can be administered over a long period. Another object is to provide a method for production of the interleukin production regulator. Further object is to provide a pharmaceutical composition and a food, each of which comprises the interleukin production regulator.

Disclosed are: a method for producing an interleukin production regulator having both an effect of maintaining or promoting the production of interleukin-10 and an effect of maintaining or inhibiting the production of interleukin-12, comprising the step of disrupting a cell of a microorganism belonging to the genus Bifidobacterium; an interleukin production regulator produced by the method; and a pharmaceutical and a food, each comprising the interleukin production regulator.

### TECHNICAL FIELD

The present invention relates to an interleukin production regulator having the effect of maintaining or promoting the production of interleukin-10 and the effect of maintaining or inhibiting the production of interleukin-12, a pharmaceutical composition and food or drink containing the interleukin production regulator, and a method for producing the interleukin production regulator.

### BACKGROUND ART

The main symptom of autoimmune disease such as rheumatic disease or inflammatory bowel disease is inflammation. It is believed that there are cytokines that cause inflammation and the occurrence of inflammation depends on the balance between cytokines sending signals and cytokine receptors receiving the signals. Further, it is believed that inflammation spontaneously heals when anti-inflammatory cytokines are stronger, but when the action of proinflammatory cytokines is strong, inflammation continues for a long period of time and becomes chronic inflammation. As described above, since it is believed that the balance of production of cytokines (e.g., interleukins) significantly contributes to the development of autoimmune disease, it can be considered that regulation of the production of these cytokines is important to prevent or treat such disease.

IL-10 is a cytokine produced by T cells, B cells, monocytes, and macrophages. IL-10 promotes the proliferation and differentiation of B cells into antibody-secreting cells, and mostly exhibits anti-inflammatory activities. It is believed that this is based on a mechanism where IL-10 upregulates IL-1RA expression by monocytes and inhibits the majority of monocyte inflammatory activities. It has already become apparent that IL-10 inhibits the production of intestinal collagenase and type IV collagenase by interfering with a PGE2-cAMP-dependent pathway, and therefore it is believed that IL-10 can be used as a regulator of connective tissue destruction seen in chronic inflammatory disease. Patent Document 3 discloses Lactococcus strains (especially, Lactococcus lactis subsp. cremoris C60) as microorganisms which efficiently induce the production of IL-10.

IL-12 is a cytokine produced mainly by antigen presenting cells such as macrophages early in the inflammatory cascade, and is a heterodimetric protein of 70 kD composed of 35 kD and 40 kD proteins. IL-12 is a strong inducer of IFN-γ production and is also an activator of natural killer cells. It is believed that IL-12 is an important cytokine for generation of cell-mediated, or Th1, immune responses mainly through its ability to activate cells for IFN-γ production. That is, IL-12 generally exhibits proinflammatory activities.

It is known that bifidobacteria exhibit various physiological effects based on their functionality, such as regulation of intestinal functions and reduction of serum cholesterol, when taken into the body in the form of, for example, fermented milk. In recent years, the concept of “probiotics (live microorganisms which beneficially affect host health maintenance)” has been introduced, and therefore such physiological effects of bifidobacteria are receiving broad attention in reflection of consumer health consciousness, and various studies are being conducted. The word “probiotics” was originally defined as “live microbial feed supplements which beneficially affect the host animal by improving its intestinal microbial flora balance”, but is now often used in the broad sense described above.

Among various physiological effects of bifidobacteria, effects on autoimmune disease such as insulin-dependent diabetes and chronic rheumatoid arthritis, irritable bowel syndrome, and inflammatory bowel disease such as ulcerative colitis and Crohn's disease have begun to receive attention. In this regard, for example, a therapeutic agent for ulcerative colitis containing the cells of a bifidobacterium as an active ingredient (see Patent Document 1) and Bifidobacterium strains effective for prevention and/or treatment of gastrointestinal inflammatory activity such as inflammatory bowel disease or irritable bowel syndrome (see Patent Document 2) have already been known.

Further, it has been already reported that the cells of bifidobacteria stimulate professional antigen presenting cells such as macrophages and dendritic cells to induce IL-12 production (i.e., an IL-12 production-inducing ability) (see Non-Patent Document 1 and Patent Document 2). Further, it has been already reported that bifidobacteria induce IL-10 production (i.e., an IL-10 production-inducing ability) (see Patent Document 2). It is believed that the ability of bifidobacteria to induce the production of various interleukins (ILs) varies depending on the kind of strain, and various studies have been conducted until now based on such a way of thinking.
Patent Document 1: Japanese Patent Application Laid-Open No. 7-126177
Patent Document 2: Japanese Patent Application National Publication No. 2005-508617
Patent Document 3: Japanese Patent Application Laid-Open No. 2005-154387
Non-Patent Document 1: International Archives of Allergy and Immunology (Int Arch Allergy Immunol), vol. 135, pp. 205-215, 2004

### DISCLOSURE OF THE INVENTION

As described above in the Background Art, it is important to regulate the production of cytokines, especially interleukins, for prevention, treatment, and prevention of recurrence of intestinal disease and autoimmune disease accompanied by inflammation as a main symptom. Among various cytokines, IL-10 as an anti-inflammatory cytokine and IL-12 as a proinflammatory cytokine can be considered important.

The present inventors have reached a conception that the prevention, treatment, and prevention of recurrence of inflammatory symptoms can be effectively achieved by increasing the production quantity of IL-10 as an anti-inflammatory cytokine while simultaneously decreasing the production quantity of IL-12 as a proinflammatory cytokine. More specifically, the present inventors have reached a conception that the prevention, treatment, and prevention of recurrence of inflammatory symptoms can be effectively achieved by an interleukin production regulator having both the effect of maintaining or promoting the production of interleukin-10 and the effect of maintaining or inhibiting the production of interleukin-12.

Complete cures for intestinal disease and autoimmune disease accompanied by inflammation as a main symptom have not yet been established. One of the reasons for this is the fact that the real causes of such diseases have not yet been determined. For this reason, therapeutic strategies for these diseases aim at inducing remission (i.e., temporary relief of symptoms) early and maintaining such a state as long as possible, rather than at achieving cure (i.e., complete disappearance of symptoms). Further, interruption of drug administration leads to exacerbation (recurrence) of symptoms, and therefore drug administration needs to be continued for a long period of time. Also in the case of aiming at prevention of inflammatory symptoms, drug administration needs to be continued for a long period of time. Therefore, it is particularly strongly desired that drugs to be used for prevention, treatment, and prevention of recurrence of inflammatory symptoms are highly safe and can be administered for a long period of time without fear.

It is therefore an object of the present invention to provide an interleukin production regulator which can be used for prevention, treatment, and prevention of recurrence of intestinal disease and autoimmune disease accompanied by inflammation as a main symptom and which has both the effect of maintaining or promoting the production of interleukin-10 and the effect of maintaining or inhibiting the production of interleukin-12, and a method for producing such an interleukin production regulator. Further, the present invention provides a pharmaceutical composition containing the interleukin production regulator as an active ingredient and a food or drink containing the interleukin production regulator.

It is also an object of the present invention to provide an interleukin production regulator which can be used for prevention, treatment, and prevention of recurrence of intestinal disease and autoimmune disease accompanied by inflammation as a main symptom, which is highly safe and can be administered for a long period of time without fear, and a method for producing such an interleukin production regulator. The present invention further provides a pharmaceutical composition containing the interleukin production regulator as an active ingredient and a food or drink containing the interleukin production regulator.

In order to achieve the above objects, the present inventors have searched a raw material which can be used for prevention, treatment, and prevention of recurrence of intestinal disease and autoimmune disease accompanied by inflammation as a main symptom, which is highly safe when used as a pharmaceutical preparation or food or drink, and can be consumed for a long period of time without fear. As a result, they have found that a cell disruption product obtained by carrying out the step of disrupting a microorganism belonging to the genus Bifidobacterium has an interleukin production-regulating ability, having simultaneously the effect of maintaining or promoting the production of interleukin-10 and the effect of maintaining or inhibiting the production of interleukin-12, and this finding has led to the completion of the present invention.

Amazingly, the present inventors have also found that the cell disruption products of any test microorganisms (as starting materials) belonging to the genus Bifidobacterium obtained by carrying out the step of disrupting a microorganism belonging to the genus Bifidobacterium have such useful interleukin production-regulating ability. That is, such a useful interleukin production-regulating ability can be obtained from a cell disruption product of any microorganism belonging to the genus Bifidobacterium obtained by sufficiently disrupting the cells thereof.

Such amazing findings disprove common knowledge among those skilled in the art which is a premise of the prior art documents such as the Non-Patent Document 1, that is, the common knowledge that the ability of microorganisms belonging to the genus Bifidobacterium to induce the production of various interleukins varies depending on the kind of strain, and therefore in order to use a desired interleukin production-inducing ability, it is necessary to conduct research and development to search a desired strain by screening various strains, and the use of (the cells of a strain having) a desired interleukin production-inducing ability can be realized only after carrying out such screening.

The present invention is directed to a method for producing an interleukin production regulator having the effect of maintaining or promoting the production of interleukin-10 and the effect of maintaining or inhibiting the production of interleukin-12, the method comprising the step of disrupting a microorganism belonging to the genus Bifidobacterium.

In the production method according to the present invention, the interleukin production regulator preferably achieves a ratio of the production quantity of interleukin-10 acceleratively produced by its effect of maintaining or promoting the production of interleukin-10 to the production quantity of interleukin-12 suppressively produced by its effect of maintaining or inhibiting the production of interleukin-12 (i.e., a ratio of interleukin-10/interleukin-12) of 10 or more, more preferably 20 or more.

Further, the step of disrupting a microorganism is preferably carried out by physical disruption, more preferably by ultrasonic disruption.

Further, the microorganism belonging to the genus Bifidobacterium is preferably one or more microorganism selected from the group consisting of microorganisms belonging to the species Bifidobacterium longum, microorganisms belonging to the species Bifidobacterium angulatum, microorganisms belonging to the species Bifidobacterium pseudocatenulatum, and microorganisms belonging to the species Bifidobacterium catenulatum.

The present invention is also directed to an interleukin production regulator obtained by the production method described above, the interleukin production regulator having the effect of maintaining or promoting the production of interleukin-10 and the effect of maintaining or inhibiting the production of interleukin-12.

The interleukin production regulator according to the present invention preferably achieves a ratio of the production quantity of interleukin-10 acceleratively produced by its effect of maintaining or promoting the production of interleukin-10 to the production quantity of interleukin-12 suppressively produced by its effect of maintaining or inhibiting the production of interleukin-12 (i.e., a ratio of interleukin-10/interleukin-12) of 10 or more, more preferably 20 or more.

The present invention is also directed to a pharmaceutical composition including the interleukin production regulator described above as an active ingredient.

The present invention is also directed to a pharmaceutical composition including the interleukin production regulator described above as an active ingredient and a pharmaceutically acceptable carrier.

The present invention is also directed to a pharmaceutical composition for preventing and/or treating autoimmune disease or intestinal disease, comprising the interleukin production regulator described above as an active ingredient.

The present invention is also directed to a pharmaceutical composition for preventing and/or treating autoimmune disease or intestinal disease, comprising the interleukin production regulator described above as an active ingredient and a pharmaceutically acceptable carrier.

The present invention is also directed to the use of a microorganism belonging to the genus Bifidobacterium to produce the interleukin production regulator described above.

The present invention is also directed to the use of the interleukin production regulator described above to produce the pharmaceutical composition described above.

The present invention is also directed to a method for simultaneously maintaining or promoting the production of interleukin-10 and maintaining or inhibiting the production of interleukin-12 of a mammal (including human) by administering the interleukin production regulator described above.

The present invention is also directed to a method for preventing and/or treating autoimmune disease or intestinal disease of a mammal (including human) by administering the pharmaceutical composition described above.

In a preferred aspect, the autoimmune disease is insulin-dependent diabetes (type 1 diabetes) or chronic rheumatoid arthritis.

In a preferred aspect, the intestinal disease is peptic colitis, Crohn's disease, intractable inflammatory bowel disease, or irritable bowel syndrome.

The present invention is also directed to a food or drink including the interleukin production regulator described above.

The present invention is also directed to a food additive including the interleukin production regulator described above.

The present invention is also directed to a food or drink for preventing and/or treating autoimmune disease or intestinal disease, comprising the interleukin production regulator described above.

The present invention is also directed to the use of the interleukin production regulator to produce the food or drink described above.

The present invention is also directed to a method for preventing and/or treating autoimmune disease or intestinal disease of a mammal (including human) by administering the food or drink described above.

In a preferred aspect, the auto immune disease is insulin-dependent diabetes (type 1 diabetes) or chronic rheumatoid arthritis.

In a preferred aspect, the intestinal disease is peptic colitis, Crohn's disease, intractable inflammatory bowel disease, or irritable bowel syndrome.

In a preferred aspect, the food or drink is a health food, functional food, enteral food, food for special dietary use, food with nutrient function claims, food for specified health uses, or qualified food for specified health uses.

The present invention is also directed to a feed including the interleukin production regulator described above.

According to the present invention, it is possible to produce an interleukin production regulator which can be used for prevention, treatment, and prevention of recurrence of intestinal disease and autoimmune disease accompanied by inflammation as a main symptom, which has both the effect of maintaining or promoting the production of interleukin-10 and the effect of maintaining or inhibiting the production of interleukin-12. Further, it is also possible to provide such an interleukin production regulator, a pharmaceutical composition containing the interleukin production regulator as an active ingredient, and a food or drink containing the interleukin production regulator.

The interleukin production regulator according to the present invention is useful for prevention, treatment, and prevention of recurrence of intestinal disease and autoimmune disease accompanied by inflammation as a main symptom, that is, autoimmune disease such as insulin-dependent diabetes and chronic rheumatoid arthritis, irritable bowel syndrome, and inflammatory bowel disease such as ulcerative colitis and Crohn's disease.

The interleukin production regulator, pharmaceutical composition and food or drink according to the present invention use as a starting material, the cells of a microorganism belonging to the genus Bifidobacterium, that is, a so-called bifidobacterium. Bifidobacteria have been historically consumed by humans in the form of, for example, yogurt for a very long period of time, and therefore their safety for humans is assured at a very high level. For this reason, the interleukin production regulator, pharmaceutical composition and food or drink according to the present invention are extremely safe, and therefore can be administered or consumed for a long period of time without fear.

### BEST MODE FOR CARRYING OUT THE INVENTION

Hereinbelow, a preferred embodiment of the present invention will be described in detail. However, the present invention is not limited to the following preferred embodiment, and changes can be freely made within the scope of the invention. It is to be noted that “percent” herein refers to “percent by mass” unless otherwise specified.

An interleukin production regulator, pharmaceutical composition and food or drink according to the present invention contains a cell disruption product of a microorganism belonging to the genus Bifidobacterium as an active ingredient. The phrase “contains a cell disruption product of a microorganism belonging to the genus Bifidobacterium as an active ingredient” herein means that a cell disruption product of a microorganism belonging to the genus Bifidobacterium is contained in an effective amount to obtain a desired effect (i.e., an interleukin production-regulating ability having simultaneously the effect of maintaining or promoting the production of interleukin-10 and the effect of maintaining or inhibiting the production of interleukin-12).

That is, the interleukin production regulator, pharmaceutical composition and food or drink according to the present invention containing a cell disruption product of a microorganism belonging to the genus Bifidobacterium enjoys an interleukin production-regulating ability characterized by the effect of maintaining or promoting the production of interleukin-10 and the effect of maintaining or inhibiting the production of interleukin-12.

A strain of the microorganism belonging to the genus Bifidobacterium to be used in the present invention is not particularly limited, and may be one previously deposited as a strain belonging to the genus Bifidobacterium in a public culture collection such as the American Type Culture Collection (ATCC), Japan Collection of Microorganisms (JCM), Northeast Texas Community College (NTCC), or Deutsche Sammlung von Mikroorganismen und Zellkulturen (DSM) or may be one isolated from nature (e.g., from human feces) by such a well-known method as described above. Examples of a strain deposited in public culture collections include FERM BP-7787, ATCC 27535, JCM 7041, and JCM 1194.

As a supply source of the microorganism belonging to the genus Bifidobacterium, a material containing such a microorganism can be used. Examples of such a material include cell suspensions, cell cultures (including cells, culture supernatant, and culture medium components), cell culture solutions obtained by removing solid matter from cell cultures, freeze-dried cell suspensions, freeze-dried cell cultures, and fermented milk using a food or drink fermented by bifidobacteria, such as a drink containing bifidobacteria, acidified milk, and yogurt. The microorganism may be isolated from such a material, or such a material containing the microorganism may be directly used.

The microorganism belonging to the genus Bifidobacterium does not always need to be a single Bifidobacterium strain, and plural strains of the microorganism belonging to the genus Bifidobacterium may be used in combination. Further, the microorganism belonging to the genus Bifidobacterium can be appropriately used in the form of viable cells, wet cells, dried cells, or dead cells.

An active ingredient of the interleukin production regulator according to the present invention is a cell disruption product of a microorganism belonging to the genus Bifidobacterium. The step of disrupting a microorganism belonging to the genus Bifidobacterium included in the method for producing an interleukin production regulator according to the present invention is preferably carried out by physical disruption. Physical disruption is particularly advantageous because, for example, it is not necessary to add any additional substance and therefore safety and security are not impaired. A specific example of a means for disrupting a microorganism includes physical disruption using a French press or a cell disrupter (e.g., Fast Prep FP120 manufactured by Funakoshi Corporation). Preferably, an ultrasonic disrupter (e.g., BRANSON SONIFIER 450) is used. In this case, physical disruption can be accomplished by ultrasonic disruption treatment at an output of about 35 W (in a case where the amount of a sample suspension is about 4 mL) for 5 minutes or longer (preferably 10 minutes or longer, more preferably 15 minutes or longer and generally 60 minutes or shorter). Preferred physical disruption can be accomplished also by ultrasonic treatment carried out by giving energy equal to that described above per unit volume. For example, preferred physical disruption can be accomplished by carrying out such treatment that an energy of generally 2,600 joules (J) or more, preferably 5,200 joules or more, more preferably 7,800 joules or more and usually 31,500 joules or less is given per milliliter of a sample solution. An ultrasonic frequency to be used in the ultrasonic treatment is generally in the range of 10 to 50 kHz, preferably in the range of 15 to 40 kHz, particularly preferably in the range of 15 to 30 kHz. Examples of a device to be used for the ultrasonic treatment include, in addition to the BRANSON SONIFIER described above, TITEC VP-5S, VP-15S, and VP-30S and MISONIX ASTRASON S3000 and XL2020, for example. A larger output of the ultrasonic disrupter tends to be able to sufficiently disrupt a microorganism in a shorter time. The step of disrupting a microorganism can be achieved by such ultrasonic treatment as described above or a comparable physical disruption method.

The cell disruption product of a microorganism belonging to the genus Bifidobacterium contained as an active ingredient in the interleukin production regulator, pharmaceutical composition and food or drink according to the present invention is a natural product and is therefore highly safe when consumed, and is contained in some food products and daily consumed, and has no toxicity, and produces few side effects even when continuously consumed for a long period of time. Therefore, the cell disruption product of a microorganism belonging to the genus Bifidobacterium can be appropriately administered via, for example, the oral route, and can be formed into tablets, capsules, troches, syrups, granules, powders, and the like by known methods. Further, the cell disruption product of a microorganism belonging to the genus Bifidobacterium can be added as an active ingredient to a food product, and can also be processed into a functional food having the effect of preventing and/or treating autoimmune disease or intestinal disease as one aspect of prevention and/or treatment of autoimmune disease or intestinal disease.

The dose of the cell disruption product of a microorganism belonging to the genus Bifidobacterium as an active ingredient of the interleukin production regulator, pharmaceutical composition and food or drink according to the present invention varies depending on dosage form, symptom, age, body weight, etc., but is in the range of 0.1 μg to 0.5 g/kg body weight/day via oral route, preferably in the range of 1 μg to 0.2 g/kg body weight/day, particularly preferably in the range of 10 μg to 50 mg/kg body weight/day in order to effectively obtain its effect of preventing and/or treating autoimmune disease or intestinal disease.

The pharmaceutical composition according to the present invention can be produced by, for example, preparing the cell disruption product of a microorganism belonging to the genus Bifidobacterium using any pharmaceutically acceptable additives such as excipients. In the case of preparing such a formulation, the amount of the cell disruption product of a microorganism belonging to the genus Bifidobacterium contained in the formulation is usually in the range of 0.001 to 10% by mass, preferably in the range of 0.01 to 10% by mass. Examples of the additives to be used for preparing the formulation include excipients, binders, disintegrants, lubricants, stabilizers, flavoring agents, diluents, and injectable solvents.

Examples of the excipients include: sugar derivatives such as lactose, sucrose, glucose, mannitol, and sorbitol; starch derivatives such as corn starch, potato starch, α-starch, dextrin, and carboxymethyl starch; cellulose derivatives such as crystalline cellulose, hydroxypropyl cellulose, hydroxypropylmethyl cellulose, carboxymethyl cellulose, and carboxymethylcellulose calcium; gum arabic; dextran; pullulan; silicate derivatives such as light anhydrous silicic acid, synthetic aluminum silicate, and magnesium aluminometasilicate; phosphate derivatives such as calcium phosphate; carbonate derivatives such as calcium carbonate; and sulfate derivatives such as calcium sulfate. Examples of the binders include, in addition to the excipients mentioned above, gelatin; polyvinyl pyrrolidone; and magrogol. Examples of the disintegrants include, in addition to the excipients mentioned above, chemically-modified starch or cellulose derivatives such as crosscarmellose sodium, carboxymethylstarch sodium, and cross-linked polyvinyl pyrrolidone. Examples of the lubricants include talc; stearic acid; metal stearates such as calcium stearate and magnesium stearate; colloidal silica; waxes such as bee gum and spermaceti wax; boric acid; glycol; carboxylic acids such as fumaric acid and adipic acid; sodium carboxylates such as sodium benzoate; sulfates such as sodium sulfate; leucine; lauryl sulfates such as sodium lauryl sulfate and magnesium lauryl sulfate; silicates such as silicic acid anhydride and silicic acid hydrate; and starch derivatives. Examples of the stabilizers include p-hydroxybenzoic esters such as methylparaben and propylparaben; alcohols such as chlorobutanol, benzyl alcohol, and phenylethyl alcohol; benzalkonium chloride; acetic anhydride; and sorbic acid. Examples of the flavoring agents include sweeteners, acidulants, and flavors. Examples of the injectable solvents include water, ethanol, and glycerin.

Examples of administration route of the pharmaceutical composition according to the present invention include oral administration and non-oral administration such as enteral administration. Examples of dosage form of the pharmaceutical composition according to the present invention include sprays, capsules, tablets, granules, syrups, emulsions, suppositories, injections, ointments, and tapes. The pharmaceutical composition according to the present invention may be blended to a food or drink or feed when administered.

The food or drink according to the present invention means a food or drink for preventing and/or treating autoimmune disease or intestinal disease containing the cell disruption product of a microorganism belonging to the genus Bifidobacterium as an active ingredient, and examples of such a food or drink include foods or drinks which can be daily consumed.

Such a food or drink can be produced by, for example, blending into the cell disruption product of a microorganism belonging to the genus Bifidobacterium, any one of sugars such as dextrin and starch; proteins such as gelatin, soy protein, and corn protein; amino acids such as alanine, glutamine, and isoleucine; polysaccharides such as cellulose and gum arabic; and oils and fats such as soybean oil and medium-chain triglyceride. Examples of the form of the food or drink include: beverages such as soft drinks, carbonated drinks, energy drinks, fruit drinks, and lactic acid drinks (concentrated stock solutions and conditioning powders of these drinks may also be included); frozen desserts such as ice creams, sorbets, and shaved ice flavored with syrups; noodles such as Japanese noodles of buckwheat, Japanese noodles of wheat, bean-starch noodles, pasta-wrappings for Chinese stuffed dumplings, pasta-wrappings for Chinese steamed dumplings, Chinese noodles, and instant noodles; confectionery such as drops, chewing gums, candies, gums, chocolates, tablets, snacks, biscuits, jellies, jams, creams, and baked goods; fishery and livestock processed foods such as steamed fish pastes, hams, and sausages; milk products such as processed milk and fermented milk; oils, fats, and oil and fat processed foods such as cooking oils, deep-frying oils, margarine, mayonnaise, shortening, whipped cream, and dressings; seasonings such as sauces and dips; soups, stews, salads, prepared foods, pickles, and bread; enteral nutritional foods; and functional foods.

The pharmaceutical composition and food or drink according to the present invention may be used singly or in combination with another pharmaceutical composition and food or drink effective on autoimmune disease or intestinal disease. Combination use of them makes it possible to enhance the effect of preventing and/or treating autoimmune disease or intestinal disease. Another pharmaceutical composition or food or drink to be used in combination with the pharmaceutical composition or food or drink according to the present invention may be added as an active ingredient to the pharmaceutical composition or food or drink according to the present invention, or may also be commercialized as a separate drug or food or drink without being added to the pharmaceutical composition or food or drink according to the present invention and sold in combination with the pharmaceutical composition or food or drink according to the present invention.

The food or drink according to the present invention can be used for various purposes utilizing the effect of preventing and/or treating autoimmune disease or intestinal disease.

The food or drink according to the present invention is preferably sold as a food or drink with an indication indicating that its use is to improve autoimmune disease or intestinal disease, for example, “food or drink having the effect of improving autoimmune disease or intestinal disease with an indication for improving autoimmune disease or intestinal disease”, “food or drink containing the cell disruption product of a microorganism belonging to the genus Bifidobacterium with an indication for improving autoimmune disease or intestinal disease”, or “food or drink containing the cell disruption product of a microorganism belonging to the genus Bifidobacterium with an indication for preventing autoimmune disease or intestinal disease”.

In this regard, it is to be noted that a phrase to be used for such an indication as described above is not limited to the phrase “for improving autoimmune disease or intestinal disease” or “for preventing autoimmune disease or intestinal disease”, and it goes without saying that any other phrases are also included in the scope of the present invention as long as they can express the effect of preventing or treating autoimmune disease or intestinal disease. As such a phrase, for example, an indication based on various uses allowing consumers to recognize the effect of preventing and/or improving autoimmune disease or intestinal disease is also possible.

The term “indication” includes all actions for informing consumers about the above-described use, and any indications reminding consumers of the above-described use or allowing consumers to know the above-described use by analogy fall in the scope of the “indication” according to the present invention irrespective of the purpose, content, object, medium, etc. of the indication. However, the indication is preferably given using expression allowing consumers to directly recognize the above-described use. Specific examples of the indication include actions of indicating the above-described use on commercial products relating to the food or drink according to the present invention or packages of the commercial products, actions of assigning, delivering, displaying for the purpose of assigning or delivering, or importing the commercial products or packages thereof with an indication indicating the above-described use, and actions of indicating the above-described use on advertisements, price lists, or business papers relating to the commercial products and displaying or distributing them, or indicating the described-above use on information including them as contents and providing the information by an electromagnetic method (e.g., Internet).

The indication is preferably one approved by the government (e.g., an indication indicated in a form based on an approval granted on the basis of any one of various systems established by the government), and such an indication is preferably attached to packages, containers, advertising media used at sales sites, such as leaflets, brochures, and POPs, and other documents.

Other examples of the indication include indications as health food, functional food, enteral food, food for special dietary use, food with health claims, food for specified health uses, food with nutrient functional claims, and quasi-drug. Particularly, indications approved by the Ministry of Health, Labor, and Welfare, such as indications approved based on the system of food for specified health uses and similar systems can be mentioned. Examples of the latter include indications as food for specified health uses, indications as qualified food for specified health uses, indications of influence on body structures or functions, and indications of reduction of disease risk claims. More specifically, typical examples thereof include indications as food for specified health uses (especially, indications of use for health) provided in the enforcement regulations of Health Promotion Law (Ministerial ordinance No. 86, Ministry of Health, Labor and Welfare, Japan, Apr. 30, 2003) and similar indications.

The pharmaceutical composition or food or drink according to the present invention can be used to maintain or promote, preferably promote the production of interleukin-10 (IL-10) as an anti-inflammatory cytokine and to maintain or inhibit, preferably inhibit the production of interleukin-12 (IL-12) as a proinflammatory cytokine in a living body, and is therefore preferably used for prevention, treatment, and prevention of recurrence of diseases involving these interleukins, such as autoimmune disease (e.g., insulin-dependent diabetes, chronic rheumatoid arthritis), irritable bowel syndrome, and inflammatory bowel disease (e.g., ulcerative colitis, Crohn's disease).

An example of the indicator of an interleukin production-regulating ability for use in the present invention includes one obtained by simultaneously evaluating the effect of maintaining or promoting the production of interleukin-10 and the effect of maintaining or inhibiting the production of interleukin-12. More specifically, an interleukin production-regulating ability can be expressed as a quantitative ratio of interleukin-10 to interleukin-12 (i.e., a ratio of IL-10/IL-12). The quantitative ratio of interleukin-10 to interleukin-12 (i.e., the ratio of IL-10/IL-12) can be calculated by, for example, a method described in PNAS, vol. 102. No. 29, pp. 10321 to 10326, 2005.

In a case where the production of interleukin-10 is maintained or promoted and the production of interleukin-12 is maintained or inhibited, the quantitative ratio of interleukin-10/interleukin-12 becomes relatively high (however, a case where the production of IL-10 and the production of IL-12 are both simultaneously maintained is not included). In this regard, it is possible that the production quantity of interleukin-10 or interleukin-12 can be measured not only by an immunologic method such as ELISA but also by a known protein concentration measurement method.

The pharmaceutical composition or food or drink according to the present invention preferably achieves a quantitative ratio of interleukin-10 to interleukin-12 (i.e., a ratio of IL-10/IL-12) of 10 or more, more preferably 15 or more, particularly preferably 20 or more. The pharmaceutical composition or food or drink according to the present invention containing the cell disruption product of a microorganism belonging to the genus Bifidobacterium in an amount required to achieve such a quantitative ratio within the above range can be preferably used for prevention, treatment, and prevention of recurrence of autoimmune disease such as insulin-dependent diabetes and chronic rheumatoid arthritis, irritable bowel syndrome, and inflammatory bowel disease such as ulcerative colitis and Crohn's disease.

### BRIEF DESCRIPTION OF THE DRAWINGS

FIG. 1 shows graphs of the production quantity of IL-12p70 induced by intact cells or a disrupted-cell product of each strain;

FIG. 2 shows graphs of the production quantity of IL-10 induced by intact cells or a disrupted-cell product of each strain; and

FIG. 3 shows graphs of the production quantity of IL-12p70 and IL-10 induced by cell products treated with ultrasonic oscillation for different periods of time and a graph of the ratio of IL-10/IL-12.

Hereinbelow, the present invention will be described in more detail with reference to the following examples, but is not limited to these examples.

### EXAMPLE 1

### Production of Tablets Containing Cell Disruption Product of Bifidobacterium longum BP-7787

150 g of powder of cell disruption product of Bifidobacterium longum BP-7787, 100 g of lactulose powder (manufactured by Morinaga Milk Industry Co., Ltd.), 635 g of malt dextrin powder (manufactured by Matsutani Chemical Industry Co., Ltd.), 85 g of skimmed milk powder (manufactured by Morinaga Milk Industry Co., Ltd.), 1 g of stevia sweetener powder (manufactured by San-Ei Gen F.F.I., Inc.), 5 g of yogurt-flavored powder (manufactured by San-Ei Gen F.F.I., Inc.), and 24 g of a glycerin fatty acid ester powdered preparation (manufactured by Riken Vitamin Co., Ltd.) were added and uniformly mixed, and the thus obtained powder mixture was continuously tableted using a tabletting machine (manufactured by Hata Iron Works Co., Ltd.) at a tabletting speed of 12 tablets/min at a pressure of 9.8 kPa to produce 1800 tablets (about 900 g, 0.5 g per tablet) containing a cell disruption product of Bifidobacterium longum BP-7787 as a symptom-relieving agent and/or a therapeutic agent for inflammatory bowel disease and irritable bowel syndrome.

### EXAMPLE 2

10.8 kg of a whey protein hydrolysate (manufactured by Morinaga Milk Industry Co., Ltd), 36 kg of dextrin (manufactured by Showa Sangyo Co., Ltd.), and small amounts of water-soluble vitamins and minerals were dissolved in 200 kg of water to prepare an aqueous phase in a tank. At the same time, 3 kg of soybean cooking oil (manufactured by Taiyo-yushi Co., Ltd.), 8.5 kg of palm oil (manufactured by Taiyo-yushi Co., Ltd.), 2.5 kg of safflower oil (manufactured by Taiyo-yushi Co., Ltd.), 0.2 kg of lecithin (manufactured by Ajinomoto Co., Inc.), 0.2 kg of fatty acid monoglyceride (manufactured by Kao Corporation), and a small amount of oil-soluble vitamins were mixed and dissolved to prepare an oil phase. The oil phase was added to the aqueous phase contained in the tank, and then they were mixed by stirring. Then, the mixture was heated to 70° C. and then further homogenized by a homogenizer at a pressure of 14.7 MPa. Then, the homogenized mixture was sterilized at 90° C. for 10 minutes, concentrated, and spray-dried to prepare about 59 kg of an intermediate powder product. To 50 kg of the thus obtained intermediate powder product, 6.8 kg of sucrose (manufactured by Hokuren), 167 g of amino acid mixture powder (manufactured by Ajinomoto Co., Inc.), and 60 g of a cell disruption product of Bifidobacterium longum BP-7787 were added, and they were uniformly mixed to produce about 56 kg of an enteral nutrition powder containing a cell disruption product of Bifidobacterium longum BP-7787 and having the effect of preventing and/or relieving the symptoms of insulin-dependent diabetes.

Hereinbelow, the present invention will be described in detail with reference to the following test examples.

<Bifidobacteria Used in Test Examples of the Present Invention>

The species, depository, number, identifying name herein of each Bifidobacterium strain used in the test examples of the present invention were shown in Table 1.

TABLE 1

| Species | Depository | Number | Identifying name |
|---|---|---|---|
| Bifidobacterium | FERM | BP-7787 | BP-7787 |
| longum | | | |
| Bifidobacterium | ATCC | 27535 | ATCC27535 |
| angulatum | | | |
| Bifidobacterium | JCM | 7041 | JCM7041 |
| pseudocatenulatum | | | |
| Bifidobacterium | JCM | 1194 | JCM1194 |
| catenulatum | | | |

<Test Method of the Present Invention>

(Preparation of Intact Cells)

Each of the Bifidobacterium strains was cultured for 16 hours using MRS (de Man Rogasa Sharpe) medium (Difco), and then an obtained culture was washed with PBS (phosphate-buffer saline) twice, and was further washed with distilled water twice, and was then suspended in distilled water and freeze-dried. The freeze-dried suspension was suspended in PBS, and was then subjected to heat treatment at 100° C. for 30 minutes to prepare intact cells.

(Preparation of Disrupted-Cell Product)

Each of the Bifidobacterium strains shown in Table 1 was cultured for 16 hours using MRS (de Man Rogasa Sharpe) medium (Difco), and then an obtained culture was washed with PBS (phosphate-buffer saline) twice, and was further washed with distilled water twice, and was then suspended in distilled water to obtain about 4 mL of a sample. The sample was subjected to ultrasonic treatment on ice using an ultrasonic disrupter (BRANSON SONIFIER 450) for 60 minutes (output control 4, output: equivalent of about 35W, frequency: 20 kHz, constant). The sample was centrifuged at 800×g for 30 minutes to remove intact cells. The thus obtained sample was observed with a speculum to confirm that intact cells did not remain therein, and was then freeze-dried. The freeze-dried sample was suspended in PBS, and was then subjected to heat treatment at 100° C. for 30 minutes to prepare a disrupted-cell product.

(Preparation of Cell Product Treated with Ultrasonic Oscillation)

B. pseudocatenulatum JCM7041 was cultured for 16 hours using MRS (de Man Rogasa Sharpe) medium (Difco), and an obtained culture was washed with PBS (phosphate-buffer saline) twice, and was then suspended in PBS to obtain culture suspensions each having a volume of about 4 mL. The culture suspensions were subjected to ultrasonic treatment on ice using an ultrasonic disrupter (BRANSON SONIFIER 450) for 0, 5, 15, 30, and 60 minutes, respectively (output control 4, output: equivalent of about 35 W, frequency: 20 kHz, constant), and were then subjected to heat treatment at 100° C. for 30 minutes.

(Preparation of Spleen Cells)

Six-week-old male BALB/c mice (available from Charles River Laboratories) were prepared as experimental animals, and were then dissected at the age of 7 to 9 weeks to extract their spleens. Spleen cells were obtained from the dissected spleens, and were then treated with a red blood cell lysis solution (0.144 M ammonium chloride, 17 mM trisaminomethane, pH 7.65) for 3 minutes to prepare spleen cells from which red blood cells had been removed.

(Experimental Conditions)

The spleen cells prepared by the method described above with reference to “Preparation of Spleen Cells” were suspended in a medium obtained by adding 10% FCS (Gibco), 100 IU/mL of penicillin, and 0.1 mg/mL of streptomycin to RPMI1640 (SIGMA) to obtain a cell suspension containing 2×10⁶ cells per milliliter. Then, 0.5 mL of the cell suspension was mixed with the cell product so that a final concentration thereof became 1 g/mL or 10 μg/mL, and the thus obtained mixture was cultured in a 48-well microplate (FALCON) at 37° C. in the presence of 5% CO₂. After a lapse of 2 days, a culture supernatant was collected to measure cytokines contained in the culture supernatant.

(Measurement of Cytokines)

The concentration of IL-10 was measured by ELISA using Duo Set (R&D Systems). The concentration of IL-12p70 was measured by concentrating the culture supernatant to 1/10 by ultrafiltration (MultiScreen Ultrcel-10, MILLIPORE) and then performing ELISA using Quantikine (R&D Systems).

### TEST EXAMPLE 1

### Comparison of Ability to Induce Production of IL-12 and IL-10 Between Disrupted-Cell Product and Intact Cells

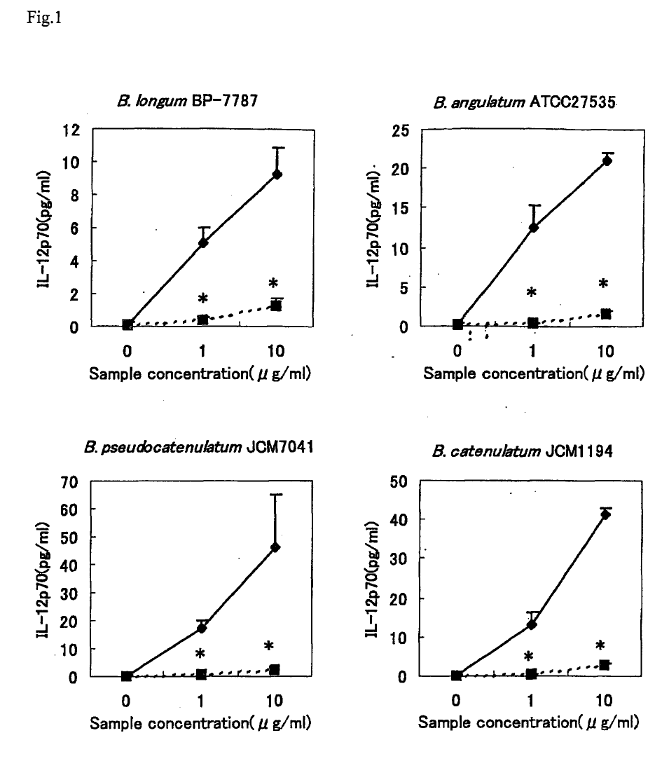

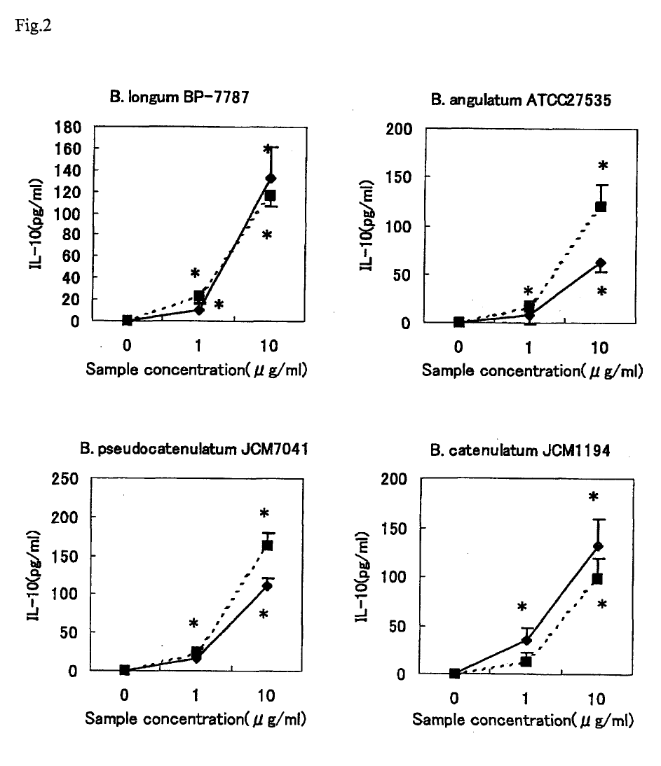

The production quantities of cytokines induced in mouse spleen cells by the intact cells or disrupted-cell product of each of the strains are shown in FIG. 1 (IL-12p70) and FIG. 2 (IL-10).

FIG. 1 shows graphs of the production quantity of IL-12p70 induced in mouse spleen cells by the addition of the intact cells or disrupted-cell product in the Test Example 1 of the present invention. In FIG. 1, averages of three experiments and standard deviations are shown. Further, the symbol “*” in FIG. 1 indicates that there was a significant difference at a significance level of 5% or less when a case where the intact cells were added (indicated by a solid line) was compared by t-test with a case where the disrupted-cell product was added (indicated by a broken line).

As a result, in all the cases of the strains used in this test, the production quantity of IL-12p70 induced by the addition of the disrupted-cell product was significantly (t-test at significance level of 5% or less) lower at some sample concentrations as compared to a case where the intact cells were added. This indicates that the ability of the disrupted-cell product to induce the production of IL-12 is lower than that of the intact cells.

FIG. 2 shows graphs of the production quantity of IL-10 induced in mouse spleen cells by the addition of the intact cells or disrupted-cell product in the Test Example 1 of the present invention. In FIG. 2, averages of three experiments and standard deviations are shown. Further, the symbol “*” in FIG. 2 indicates that there was a significant difference at a significance level of 5% or less when a case where the intact cells were added (indicated by a solid line) was compared by t-test with a case where the intact cells were not added (i.e., control) or when a case where the disrupted-cell product was added (indicated by a broken line) was compared by t-test with a case where the disrupted-cell product was not added (i.e., control).

As a result, in all the cases of the strains used in this test, the production quantity of IL-10 was significantly (t-test at significance level of 5% or less) increased by the addition of the intact cells and the disrupted-cell product as compared to a control not containing such a cell product. This indicated that both the intact cells and the disrupted-cell product have the ability to induce the production of IL-10.

The ratio of the production quantity of IL-10 to the production quantity of IL-12p70 (i.e., the ratio of IL-10/IL-12) measured after the addition of the intact cells or the disrupted-cell product of each strain at a concentration of 10 μg/mL is shown in Table 2. In this regard, it is to be noted that in a case where the concentration of IL-12p70 was a detection limit or lower (i.e., 0.25 pg/mL or lower), the production quantity ratio was calculated using the detection limit. In all the cases of the strains, the ratio of IL-10/IL-12 was greatly increased when the disrupted-cell product was added.

TABLE 2
Ratio of IL-10/IL-12 of cytokine induced by
intact cells and disrupted-cell product

| Species | Strain | Intact cells | Disrupted-cell product |
|---|---|---|---|
| Bifidobacterium | BP-7787 | 14.8 | 107.9 |
| longum | | | |
| Bifidobacterium | ATCC27535 | 3.0 | 71.9 |
| angulatum | | | |
| Bifidobacterium | JCM7041 | 2.6 | 71.6 |
| pseudocatenulatum | | | |
| Bifidobacterium | JCM1194 | 3.2 | 36.0 |
| catenulatum | | | |

As described above, the ability of the disrupted-cell products of the bifidobacteria to induce the production of IL-12 was lower than that of the intact cells of the bifidobacteria, but the disrupted-cell products and intact cells of the bifidobacteria both had the ability to induce the production of IL-10. This result indicated that the ability of the bifidobacteria to induce the production of IL-12 can be reduced by disrupting their cells while maintaining their ability to induce the production of IL-10. Further, the ratio of IL-10/IL-12 was greatly increased when the disrupted-cell product was added, compared to when the intact cells were added. This indicated that the ratio of IL-10/IL-12 can be significantly improved by disrupting the cells of the bifidobacteria.

### TEST EXAMPLE 2

### Study on Length of Cell Disruption Time

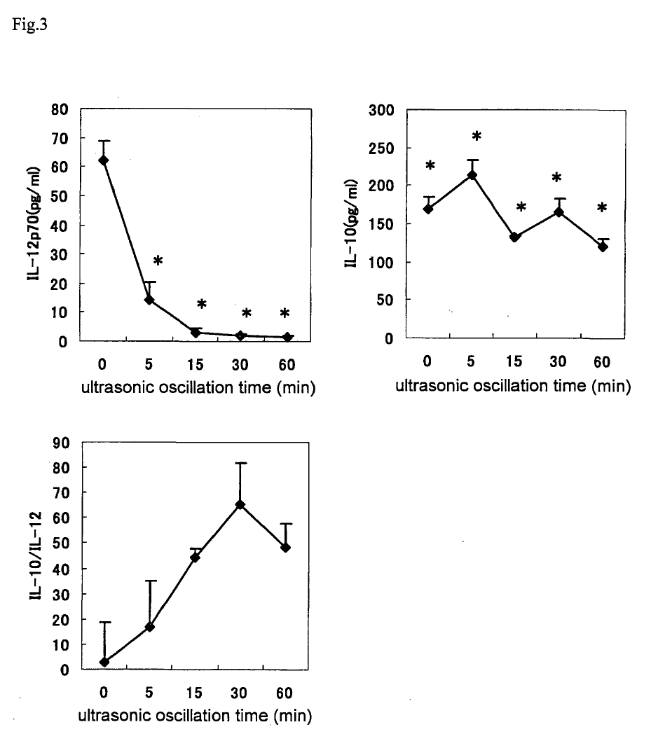

The production quantities of cytokines induced in mouse spleen cells by the addition of the cell products treated with ultrasonic oscillation for different periods of time (sample concentration: 10 μg/mL, B. pseudocatenulatum JCM7041) are shown in FIG. 3.

FIG. 3 shows graphs of the production quantity of IL-12p70 or IL-10 induced in mouse spleen cells by the addition of the cell products of B. pseudocatenulatum JCM7041 treated with ultrasonic oscillation for different periods of time shown therein and a graph of the ratio of IL-10/IL-12 (“pg/mL”/“pg/mL”) in the Test Example 2 of the present invention. In FIG. 3, averages of three experiments and standard deviations are shown. In the graph of the production quantity of IL-12p70, the symbol “*” indicates that there was a significant difference at a significance level of 0.05 or less when a case where the cell product treated with ultrasonic oscillation was added was compared by t-test with a case where the intact cells (i.e., the cell product treated with ultrasonic oscillation for 0 minute) were added. In the graph of the production quantity of IL-10, the symbol “*” indicates that there was a significant difference at a significance level of 5% or less when a case where the cell product was added was compared by t-test with a control (detection limit or less) not containing the cell product.

As a result, in all the cases where the cell products treated with ultrasonic oscillation for 5 minutes or longer were added, the production quantity of IL-12 was significantly (significance level: 5% or less) reduced, and a longer cell disruption time resulted in a smaller production quantity of IL-12. More specifically, when the cell product treated with ultrasonic oscillation for 15 minutes or longer was added, the production quantity of IL-12 was extremely reduced, and when the cell product treated with ultrasonic oscillation for 30 minutes or longer was added, almost no IL-12 was produced. On the other hand, the production quantity of IL-10 was significantly higher when any one of the cell products treated with ultrasonic oscillation for different periods of time was added as compared to a control not containing the cell product. More specifically, the production quantity of IL-10 was slightly increased by increasing the cell disruption time from 0 to 5 minutes, but after that, tended to be slightly reduced as the cell disruption time was increased. However, all the cell products treated with ultrasonic oscillation basically maintained the ability to induce the production of IL-10 irrespective of the length of cell disruption time.

The above results indicated that the ability of the bifidobacteria to induce the production of IL-12 can be reduced by treating the cells of the bifidobacteria with ultrasonic oscillation using the ultrasonic disrupter (BRANSON SONIFIER 450) for at least 5 minutes or longer. Further, as can be seen from the graph of the ratio of IL-10/Il-12 in FIG. 3, the cells of the bifidobacteria are preferably treated with ultrasonic oscillation for at least 5 minutes or longer, more preferably 15 minutes or longer and generally 60 minutes or shorter to almost completely block their ability to induce the production of IL-12 and to achieve a high ratio of IL-10/Il-12.

1. A method for producing an interleukin production regulator having the effect of maintaining or promoting the production of interleukin-10 and the effect of maintaining or inhibiting the production of interleukin-12, the method comprising the step of disrupting a microorganism belonging to the genus Bifidobacterium.

2. The production method according to claim 1, wherein the interleukin production regulator achieves a ratio of the production quantity of interleukin-10 acceleratively produced by its effect of maintaining or promoting the production of interleukin-10 to the production quantity of interleukin-12 suppressively produced by its effect of maintaining or inhibiting the production of interleukin-12 (i.e., a ratio of interleukin-10/interleukin-12) of 10 or more.

3. The production method according to claim 1, wherein the step of disrupting a microorganism is carried out by physical disruption.

4. The production method according to claim 1, wherein the step of disrupting a microorganism is carried out by ultrasonic disruption.

5. The production method according to claim 1, wherein the microorganism belonging to the genus Bifidobacterium is one or more microorganism selected from the group consisting of microorganisms belonging to the species Bifidobacterium longum, microorganisms belonging to the species Bifidobacterium angulatum, microorganisms belonging to the species Bifidobacterium pseudocatenulatum, and microorganisms belonging to the species Bifidobacterium catenulatum.

6. An interleukin production regulator obtained by the production method according to claim 1, the interleukin production regulator having the effect of maintaining or promoting the production of interleukin-10 and the effect of maintaining or inhibiting the production of interleukin-12.

7. The interleukin production regulator according to claim 6, which achieves a ratio of the production quantity of interleukin-10 acceleratively produced by its effect of maintaining or promoting the production of interleukin-10 to the production quantity of interleukin-12 suppressively produced by its effect of maintaining or inhibiting the production of interleukin-12 (i.e., a ratio of interleukin-10/interleukin-12) of 10 or more.

8. A pharmaceutical composition comprising the interleukin production regulator according to claim 6 as an active ingredient.

9. A pharmaceutical composition for preventing and/or treating autoimmune disease or intestinal disease, comprising the interleukin production regulator according to claim 6 as an active ingredient.

10. The pharmaceutical composition according to claim 9, wherein the autoimmune disease is insulin-dependent diabetes (type 1 diabetes) or chronic rheumatoid arthritis.

11. The pharmaceutical composition according to claim 9, wherein the intestinal disease is peptic colitis, Crohn's disease, intractable inflammatory bowel disease, or irritable bowel syndrome.

12. A food or drink comprising the interleukin production regulator according to claim 6.

13. A food or drink for preventing and/or treating autoimmune disease or intestinal disease, comprising the interleukin production regulator according to claim 6.

14. The food or drink according to claim 13, wherein the autoimmune disease is insulin-dependent diabetes (type 1 diabetes) or chronic rheumatoid arthritis.

15. The food or drink according to claim 13, wherein the intestinal disease is peptic colitis, Crohn's disease, intractable inflammatory bowel disease, or irritable bowel syndrome.

16. The food or drink according to claim 12, which is a health food, functional food, enteral food, food for special dietary use, food with nutrient function claims, food for specified health uses, or qualified food for specified health uses.

17. A feed comprising the interleukin production regulator according to claim 6.
